(19) United States
(12) Patent Application Publication
(10) Pub. No.: US 2004/0039301 A1
(43) Pub. Date: Feb. 26, 2004

(54) Urine collection device for a male and method therefor

(76) Inventor: A. Colleen Schmidt, Boonville, IN (US)

Correspondence Address: BOWERS HARRISON LLP, GARY K. PRICE, ESP., 25 RIVERSIDE DRIVE, PO BOX 1287, EVANSVILLE, IN, 47706-1287, US

(21) Appl. No.: 10/634,469

(22) Filed: Aug. 5, 2003

Related U.S. Application Data
(60) Provisional application No. 60/401,488, filed on Aug. 7, 2002.

(51) Int. Cl. A61B 5/00

(52) U.S. Cl. 600/580

(57) ABSTRACT

A urine collection device directed for male users who are bed-ridden in hospitals, nursing homes, at-home care patients who are totally or partially bed-ridden patients, or by other male individuals seeking the convenience of a portable urine collection device. The collection device of the present invention includes a bag having a main body portion, an upper portion, a lower portion, and an inner chamber. The bag is provided at the upper portion with an upper neck portion, and at the lower portion having a lower neck portion. The bag further includes an aperture for releasably securing or hanging the bag to an external hook. The uppermost end of the upper neck portion having an opening in fluid communication with the inner chamber of the bag. The lowermost end of the lower neck portion having an opening in fluid communication with the inner chamber of the bag. The bag further including a closure secured to the opening of the lower neck portion. The function of the closure when secured to the lower neck portion, is to preserve any fluid in the inner chamber and provide a leak resistant sealing between the lower neck portion and the closure. When the closure is released from the lower neck portion, any fluid contained in the inner chamber of the bag is allowed to flow by gravity out from the inner chamber down through the opening of the lower neck portion. The collection device further includes a hollow tube having a first end attached to the uppermost end of the upper neck portion, and a second end attached to an end of a conduit, the opposite end of the conduit sized to facilitate the penis to be inserted therein during use. Gravity allows the fluid to flow from the conduit down through the tube and to the inner chamber.

### CROSS REFERENCES TO RELATED APPLICATIONS

None.

Statement as to rights to inventions made under Federally sponsored research and development: U.S. Provisional Application for Patent 60/401,488, filed Aug. 7, 2002, with title, “Urine Collection Device for a Male” which is hereby incorporated by reference. Applicant claims priority pursuant to 35 U.S.C. Par. 119(e)(i).

### BACKGROUND OF THE INVENTION

1. Field of the Invention

This invention relates to a urine collection device for a male. More particular, the device of the present invention is directed for male users who are bed-ridden in hospitals, nursing homes, at-home care patients who are totally or partially bed-ridden patients, or by other male individuals seeking the convenience of a portable urine collection device.

2. Brief Description of Prior Art

The number of individuals using urine bottles is increasing for several reasons: an increase in the incidence of prostrate problems and other urological disorders; an ever-increasing aged population with a greater propensity for longevity and the resultant age-associated disabilities. Further, there is increased use of hand-held urinals by able-bodied males who find their use a convenience; i.e., workers at a work site without bathroom facilities, or over the road professional drivers who are between scheduled stops or are otherwise schedule pressed.

Several patents have issued that disclose various urine collection containers. Many users of these collection containers are in hospitals, nursing homes, and at-home care patients who suffer from physical disabilities that require them to be totally or partially bed-ridden. Heretofore, there has not been a suitable urine collection device that is comfortable for the user who is bed-ridden, and able to completely collect the urine from the user without spills or leakage.

The prior art containers are generally a bag for collecting the urine, said bag having an opening or cut-out for inserting the penis into the bag. It has been found difficult to maintain the opening or cut-out of the prior art container in an outstretched position. As a result, an operation of inserting the penis into the bag, or maintaining the penis in the bag during urination may be difficult.

This application in general relates to a urine collection container designed to collect a liquid urine through an elongated tube when the tube is connected to a collection bag. Gravity allows the specimen to be transferred from the tube and into the bag. As a result, the device of the present invention is comfortable for the user who is bed-ridden, and will collect the urine from the user without spills.

As will be seen from the subsequent description, the preferred embodiments of the present invention overcome shortcomings of the prior art.

### SUMMARY OF THE INVENTION

The present invention in general relates to improvements in urine collection devices. The device of the present invention is directed for male users who are bed-ridden in hospitals, nursing homes, at-home care patients who are totally or partially bed-ridden patients, or by other male individuals seeking the convenience of a portable urine collection device. The device of the present invention is comfortable for the user who is bed-ridden, and will collect the urine from the user without spills. The collection device of the present invention includes a bag having a main body portion, an upper portion, a lower portion, and an inner chamber. The bag is provided at the upper portion with an upper neck portion, and at the lower portion having a lower neck portion. The bag further includes an aperture for releasably securing or hanging the bag to an external hook. The uppermost end of the upper neck portion having an opening in fluid communication with the inner chamber of the bag. The lowermost end of the lower neck portion having an opening in fluid communication with the inner chamber of the bag. The bag further including a closure secured to the opening of the lower neck portion. The function of the closure when secured to the lower neck portion, is to retain any fluid in the inner chamber and provide a leak resistant sealing between the lower neck portion and the closure. When the closure is released from the lower neck portion, fluid contained in the inner chamber of the bag will flow by gravity out from the inner chamber down through the opening of the lower neck portion.

The collection device further includes a flexible hollow tube having a first end attached to the uppermost end of the upper neck portion, and a second end attached to an end of a conduit, the opposite end of the conduit sized to facilitate the penis to be inserted therein during use. Gravity allows the fluid to flow from the conduit down through the tube and to the inner chamber.

### BRIEF DESCRIPTION OF THE DRAWINGS

FIG. 1 is a perspective view of the present invention, a urine collection device.

FIG. 2 is an exploded sectional view of the urine collection device of FIG. 1.

### DESCRIPTION OF THE PREFERRED EMBODIMENT

In accordance with the present invention, a urine collection device for a male is disclosed. The urine collection device is directed for male users who are bed-ridden in hospitals, nursing homes, at-home care patients who are totally or partially bed-ridden patients, or by other male individuals seeking the convenience of a portable urine collection device. Specifically, it will be noted in the drawings that the urine collection device relates to a device designed to collect liquid urine through an elongated tube when the tube is connected to a collection bag. Gravity allows the urine specimen to be transferred from the tube and into the bag. In the broadest context, the urine collection device of the present invention consists of components configured and correlated with respect to each other so as to attain the desire objective.

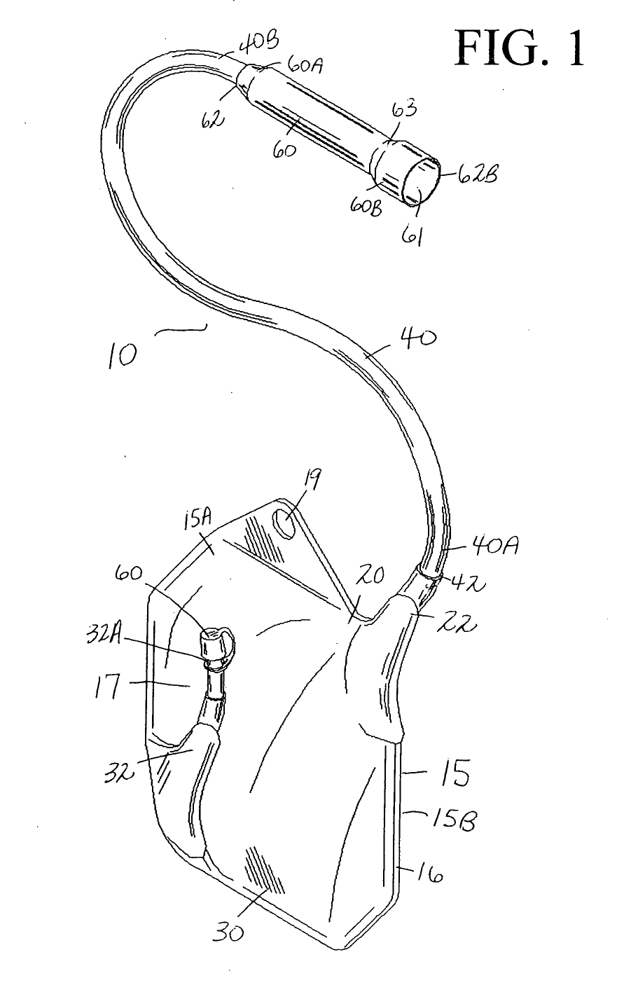

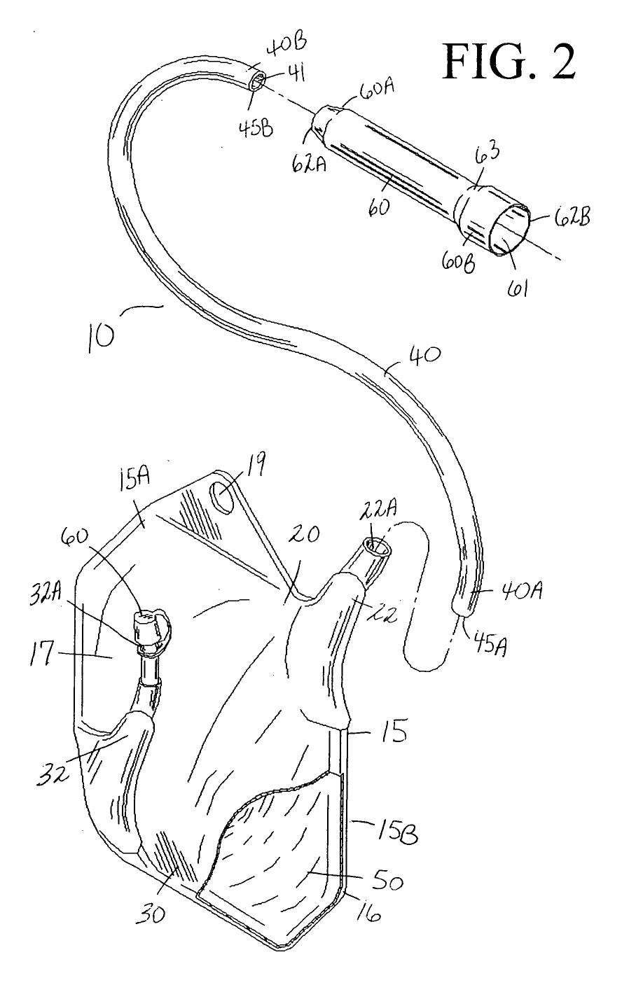

FIGS. 1-2 illustrate a preferred embodiment of a urine collection device 10 made in accordance with the present invention. As shown in the drawing, the collection device 10 includes a bag 15, the bag 15 is preferably constructed of a flexible and liquid-impervious material. The bag 15 having a generally rectangular configuration, and further includes a main body portion 17, an upper portion 20, and a lower portion 30. The bag 15 is provided at the upper portion 20 with an upper neck portion 22, and the lower portion 30 having a lower neck portion 32. The bag 15 further includes an aperture 19 preferably disposed at the upper portion 20 of the bag 15, said aperture 19 for releasably securing or hanging the bag 15 to an external hook (not shown).

The bag 15 is formed by joining a first side 15A to a second side 15B, the sides 15A, 15B defining an inner chamber 50 within the bag 15. The first side 15A is bonded to the second side 15B along a joined edge 16 longitudinally extending the circumference of the bag 15 so as to substantially describe an arc.

The uppermost end of the upper neck portion 22 includes an upper-opening 22A in fluid communication with the inner chamber 50 of the bag 15. The lowermost end of the lower neck portion 32 includes a lower-opening 32A in fluid communication with the inner chamber 50 of the bag 15. As shown in the drawing, the bag 15 further including a closure 60 releasably secured to the lower neck portion 32 and in sealing contact with the lower-opening 32A. In the preferred embodiment, the closure 60 is rotatably tightened over the lower neck portion 32, forming a liquid-tight seal therebetween. In particular, the lower neck portion 32 preferably includes an upper threaded portion (not shown) formed with threads for coupling with the closure 60; however other securing means known in the art may be used.

The function of the closure 60 when secured and in releasable locking engagement with the lower neck portion 32, is to retain any fluid in the inner chamber 50 and provide a leak resistant seal between the lower neck portion 32 and the closure 60. When the closure 60 is released from the lower neck portion 32, fluid within the inner chamber 50 is allowed to flow from the inner chamber 50, and out through the lower-opening 32A of the lower neck portion 32.

As shown in the drawing, the lower neck portion 32 is preferably disposed at the lower portion 30 of the bag 15 that represents the lowest gravitational area of the bag 15. The purpose for the lower neck portion 32 and the lower-opening 32A is to allow, when the closure 60 is released, any fluid contained within the inner chamber 50 of the bag 15 to flow by gravity from the inner chamber 50 down through the lower-opening 32A of the lower neck portion 32.

The device 10 further includes a flexible elongated tube 40 having a first end 40A, and a second end 40B opposite the first end 40A. The first end 40A is attached to the uppermost end of the upper neck portion 22 at joint 42, which joint 42 may be an integral joint of the upper neck portion 22 when manufactured. The second end 40B is attached to a conduit 60 at joint 62, which joint 62 may be an integral joint of the conduit 60 when manufactured.

The elongated tube 40 includes a longitudinally positioned interior bore 41 that extends the length of the body of the tube 40, and further includes a lower-opening 45A disposed at the first end 40A, and an upper-opening 45B disposed at the second end 40B of the tube 40. The interior bore 41 is disposed between the lower and upper openings 45A, 45B of the tube 40 so that the upper opening 45B is in fluid communication with the lower opening 45A. When the first end 40A is connected to the upper neck portion 22 as described above, the lower opening 45A of the tube 40 is fixedly centered over the upper opening 22A of the upper neck portion 22. As will be further described, in application, fluid flows through the upper opening 45B of the tube 40, through the interior bore 41 of the tube 40, down through the lower opening 45A of the tube 40, through the upper opening 22A of the upper neck portion 22, through the upper neck portion 22 and into the inner chamber 50.

The conduit 60 includes a first end 60A, and a second end 60B opposite the first end 60A. The conduit 60 includes a longitudinally positioned interior bore 61 and opposing first and second openings 62A, 62B. As shown in the drawing, the circumference of the first end 60A is preferably smaller than the circumference of the second end 60B. The first end 60A of the conduit 60 extends from the first opening 62A and terminates at a shoulder 63. The second end 60B extends from the shoulder 63 and terminates at the second opening 62B.

The first end 60A of the conduit 60 is appropriately connected to the second end 40B of the tube 40. The first opening 62A of the conduit 60 is fixedly centered over the upper-opening 45B of the tube 40, so that fluid flows through the interior bore 61 of the conduit 60, through the first opening 62A and through the upper-opening 45B, and down through the interior bore 41 of the tube 40 as discussed above. Gravity allows the fluid to flow from the conduit 60 down through the tube 40 and into the inner chamber 50.

The second end 60B of the conduit 60 is sized to facilitate a penis (not shown) to be inserted within the interior bore 61 of the conduit 60 through the second opening 62B of the second end 60B.

As shown in the drawings, the conduit 60 and the tube 40 have a generally cylindrical configuration.

Materials for the components of the urine collection device 10 may be formed of known materials. The bag 15 is preferably a unitary structure and is made of a thermoplastic material. The tube 40 and conduit 60 are preferably made of a similar thermoplastic material to resist breakage.

Application of the device 10 begins with securing the closure 60 to the opening 32A of the lower neck portion 32. The user then inserts the penis through the second opening 62B of the second end 60B of the conduit 60 so that the penis is within the interior bore 61 of the conduit 60. During use, the bag 15 should be disposed at an elevation below the second end 60B of the conduit 60. Further, the second end 60B should be disposed at an elevation above the first end 60A of the conduit 60. During the collection of the fluid, gravity, due to the conduit 60 being in an elevated position to the bag 15, causes the fluid to flow through the interior bore 61, through the first opening 62A of the conduit 60, through the upper opening 45B of the tube 40, and down through the interior bore 41 of the tube 40. Gravity allows the fluid to flow through the tube 40 and into the inner chamber 50 as discussed. As the closure 60 is secured over the lower neck portion 32, forming a liquid-tight seal therebetween, no leakage from the inner chamber 50 of the bag 15 will occur. Further, as the penis rests within the interior bore 61 of the conduit 60 during application, no leakage or spills of the fluid occurs during collection.

When collection of the fluid is completed, the fluid may be discarded from the inner chamber 50 of the device 10 by removing the closure 60 from the lower opening 32A of the lower neck portion 32. Once the closure 60 is removed, the fluid contained in the inner chamber 50 of the bag 15 flows by gravity from the inner chamber 50 out through the lower opening 32A of the lower neck portion 32.

The embodiment depicted in the drawing is intended to be merely exemplary, and is not intended to depict all possible shapes for a collection device of the present invention. Rather, bag 15 can be of any shape having mating components as described herein.

The present invention allows the collection of urine from male users who are bed-ridden in hospitals, nursing homes, at-home care patients who are totally or partially bed-ridden patients, or by other male individuals seeking the convenience of a portable urine collection device. Thus, the present invention provides safety from spills or leakage by using the flexible elongated tube 40 extending from the bag 15 as described above.

Although the description above contains many specificities, these should not be construed as limiting the scope of the invention but as merely providing illustrations of some of the presently preferred embodiments of this invention.

Thus the scope of the invention should be determined by the appended claims in the formal application and their legal equivalents, rather than by the examples given.

I claim:

1. A urine collection device for a male, said device comprising:
a bag including an upper neck portion having a upper neck opening, a lower neck portion having a lower neck opening, and a first side attached to a second side, wherein the interior surfaces of said first and second sides define an inner chamber;
an elongated tube having an interior bore that extends the length of the tube, a first end having a lower tube opening, said first end of the tube connected to the upper neck portion of the bag, and a second end having an upper tube opening, wherein the lower tube opening is fixedly centered over the upper neck opening of the upper neck portion;
wherein the tube is in fluid communication with the inner chamber;
a closure releasably secured to the lower neck portion in sealing engagement with the lower neck opening;
wherein releasing the closure from the lower neck portion allows fluid retained within the inner chamber to releasably flow through the lower neck opening;
wherein securing the closure to the lower neck portion forms a liquid tight seal so that the fluid is retained within the inner chamber.

2. The urine collection device as recited in claim 1, further comprising a conduit having a bore that extends the length of the tube, a first conduit end connected to the second end of the tube, and a second conduit end having a second conduit opening sized to facilitate a male's penis.

3. The urine collection device as recited in claim 2, wherein the first conduit end having a first conduit opening, wherein the first conduit opening is fixedly centered over the upper tube opening of the second end of the tube.

4. The urine collection device as recited in claim 1, further comprising an aperture disposed on an upper portion of the bag.

5. The urine collection device as recited in claim 1, wherein the lower neck portion is disposed at a lowest gravitational area of the bag so that retained fluid within the inner chamber flows by gravity from the inner chamber through the lower neck opening.

6. The urine collection device as recited in claim 1, wherein the bag is constructed of a liquid-impervious material.

7. The urine collection device as recited in claim 1, wherein the bag is constructed of a thermoplastic material.

8. The urine collection device as recited in claim 1, wherein the tube is constructed of a flexible thermoplastic material.

9. A urine collection device for a male, said device comprising:
a bag including an inner chamber and an outlet, said outlet in fluid communication with the inner chamber;
an elongated tube in fluid communication with the inner chamber,
a closure releasably secured to the outlet;
wherein releasing the closure from the outlet allows fluid retained within the inner chamber to releasably flow from the outlet;
wherein securing the closure to the outlet forms a liquid tight seal so that the fluid is retained within the inner chamber.

10. The urine collection device as recited in claim 9, further comprising an aperture disposed on an upper portion of the bag.

11. The urine collection device as recited in claim 9, wherein the tube further includes a first end having a lower tube opening and a second end having an upper tube opening, said first end is connected to an upper neck portion of the bag.

12. The urine collection device as recited in claim 11, further comprising a conduit having a bore that extends the length of the tube, a first conduit end connected to the second end of the tube, and a second conduit end having a second conduit opening sized to facilitate a male's penis.

13. The urine collection device as recited in claim 12, wherein the first conduit end having a first conduit opening, wherein the first conduit opening is fixedly centered over the upper tube opening of the second end of the tube.

14. The urine collection device as recited in claim 9, wherein the bag is constructed of a liquid-impervious material.

15. The urine collection device as recited in claim 9, wherein the bag is constructed of a thermoplastic material.

16. The urine collection device as recited in claim 9, wherein the tube is constructed of a flexible thermoplastic material.

17. A method for collecting and discarding a male's urine, comprising the following steps in the sequence set forth:
securing a closure to an outlet of a urine collection device, said device comprising a bag having an inner chamber, an upper neck portion, and said outlet, said outlet in fluid communication with the inner chamber, the device further including a flexible elongated tube having an interior bore, a first end connected to the upper neck portion of the bag, and a second end, said tube in fluid communication with the inner chamber, and a conduit having a bore, a first conduit end connected to the second end of the tube, and a second conduit end, wherein the first conduit end having a first conduit opening fixedly centered over an upper tube opening of the second end of the tube;
inserting the male's penis in the second conduit end of the conduit;
elevating the second end of the tube above the first end;
collecting the urine within the device, wherein the urine flows down through the tube, through the upper neck portion, and into the inner chamber of the bag;
tilting the bag so that the urine within the bag flows away from the outlet in the bag;
removing the closure from the outlet;
tilting the bag so that the urine within the bag flows toward the outlet.
